(19) United States
(12) Patent Application Publication
(10) Pub. No.: US 2022/0387087 A1
(43) Pub. Date: Dec. 8, 2022

(54) FRACTURE FIXATION PLATE

(71) Applicant: Bonebridge AG, Zug (CH)

(72) Inventors: Patrick BURKI, Solothurn (CH); Andreas SCHWEIZER, Zumikon (CH); Mathias HÄFELI, Igis (CH)

(21) Appl. No.: 17/774,536

(22) PCT Filed: Nov. 7, 2019
(86) PCT No.: PCT/CH2019/000034

§ 371 Date: May 5, 2022

(51) Int. Cl. A61B 17/80

(52) U.S. Cl. CPC A61B 17/8061; A61B 17/8057

(57) ABSTRACT

The fracture fixation plate (1) is suitable for application to the volar surface of a distal radius, wherein the fracture fixation plate (1) has an elongated body section (2) which has a free proximal end (3) and a distal end (4) on which an ulna head section (5) and a radius head section (6) connect, which diverge distally from one another, wherein the two head sections (5; 6) are each independently connected to the distal end (4) of the body section (2) via a corresponding neck section (51; 61), the body section (2) and the two head sections (5; 6) are provided with a number of threaded holes (7) for receiving bone securing elements (8) and the body section (2) also has an elongate compression hole (9), and the fracture fixation plate (1) has a bone contact surface (10) and an opposing surface (11). The bone contact surface (10) is designed such that it is convex in the region of the ulna head section (5) and the bone contact surface (10) is designed as a curved surface in the region of the radius head section (6).

The invention relates to a fracture fixation plate according to the preamble of claim 1.

From EP-B 2 675 378, a fracture plate is already known, which, however, has a special geometry which is rather disadvantageous for the intended purpose. In particular, in this known plate, the bone contact surface is designed such that it is concave in the region of the ulna head section and flat in the region of the radius head section. In order to achieve the best possible bone contact between the bone contact surfaces of the head sections of the fracture plate and the surfaces of the underlying bones or bone fragments, the radius head section and preferably also the ulna head section are therefore matched to the anatomy with regard a) to the separation between the head sections, b) the elevation angle of the head sections, and c) the rotation angle about the longitudinal axes of the head sections. Therefore, a disadvantage of this known fracture plate is the complicated matching of the bone contact surfaces of the head sections, in order to establish satisfactory bone contact.

The invention seeks to provide a remedy here. The underlying aim of the invention is to create a fracture fixation plate which enables an optimal bone contact between the bone contact surfaces of the head sections and the surfaces of the underlying bones or bone fragments.

The invention achieves the stated aim with a fracture fixation plate which has the features of claim 1.

The advantages achieved by the invention should essentially be considered to be that, by means of the fracture fixation plate according to the invention:
the bone contact surface, which is designed such that it is convex, i.e., bulging, in the region of the ulna head section, ensures a stable and reliable application of the bone contact surface on the bone surface without a complicated matching of the position of the ulna head section having to be performed; and
the bone contact surface which is curved in the region of the radius head section here also ensures a stable and reliable application of the bone contact surface on the bone surface.

In a particular embodiment, the bone contact surface is designed such that it is spherically convex in the region of the ulna head section.

In an additional embodiment, the bone contact surface, in the region of the radius head section (6), is designed such that it is convex, preferably cylindrically curved.

The bone contact surface, in the region of the ulna head section, can be substantially trapezoidal, wherein the longer base of the trapezoid comes to lie distally. The bone contact surface, in the region of the radius head section, can be substantially rectangular, wherein one of the two shorter sides of the corner of the rectangle comes to lie distally.

In a particular embodiment, the notch formed between the two head sections and their neck sections is dimensioned/formed in such a manner that the flexor pollicis longus (FPL) tendon can be accommodated therein. For this purpose, the notch can be designed such that it is V-shaped and continuously widens in the distal direction.

Measured in the plane of the plate, the notch has the area F_{E} which can be at most 10%, preferably at most 6%, of the total area F_{TOT} of the fracture fixation plate, including the notch and any possible plate holes. The area F_{E} is preferably at least 10 mm², preferably at least 20 mm². The area F_{E} is preferably at most 60 mm², preferably at most 45 mm².

In an additional embodiment, the body section (2) has at least one threaded hole with a non-rotatable, positive-locking threaded hole insert made of a softer material than the material of the bone fixation plate.

The ulna head section and preferably the radius head section can each have at least one threaded hole with a non-rotatable, positive-locking threaded hole insert made of a softer material than the material of the bone fixation plate.

The ulna head section can have three threaded holes, preferably with a non-rotatable, positive-locking threaded hole insert made of a softer material than the material of the bone fixation plate.

The radius head section can have two or three threaded holes, preferably with a non-rotatable, positive-locking threaded hole insert made of a softer material than the material of the bone fixation plate.

Preferably, the convexly curved bone contact surface, in the region of the radius head section, has a curvature which extends from the distal end of the elongated body section to a distal end of the radius head section.

The invention and developments of the invention are explained in further detail below using partially diagrammatic representations of an embodiment example.

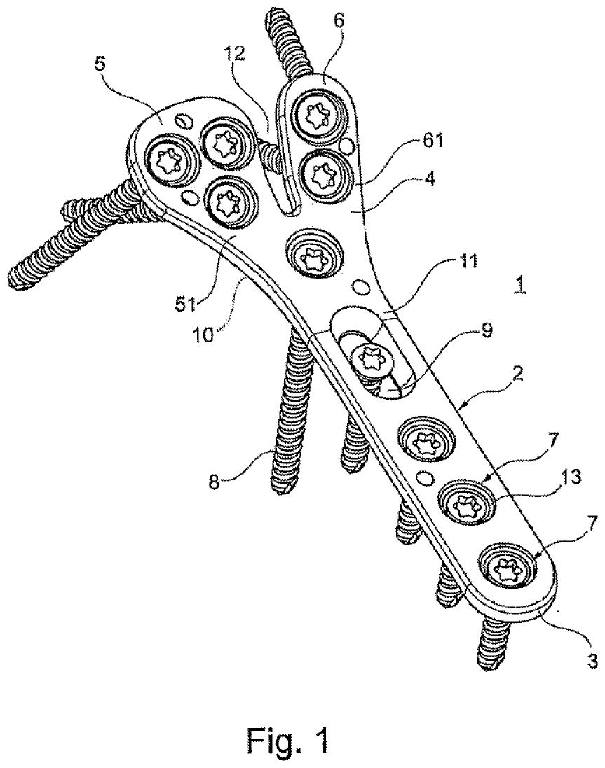

FIG. 1 shows a perspective view of an embodiment of the fracture fixation plate according to the invention;

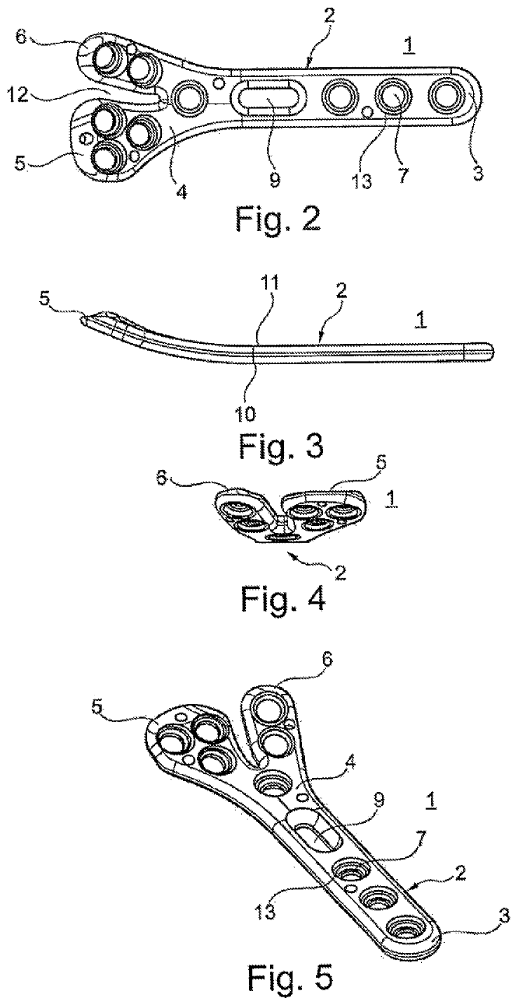

FIG. 2 shows a view onto the embodiment of the fracture fixation plate according to the invention shown in FIG. 1;

FIG. 3 shows a side view of the embodiment of the fracture fixation plate according to the invention shown in FIG. 1;

FIG. 4 shows a front view onto the ulna and radius head sections of the embodiment of the fracture fixation plate according to the invention shown in FIG. 1; and

FIG. 5 shows a perspective view of the embodiment of the fracture fixation plate according to the invention shown in FIG. 1.

The embodiment of the fracture fixation plate 1 according to the invention shown in FIG. 1-5 substantially comprises an elongated body section 2 which has a free proximal end 3 and a distal end 4, an ulna head section 5 which connects to the distal end 4, and a radius head section 6 which also connects to the distal end 4 of the body section 2.

The ulna head section 5 and the radius head section 6 diverge distally from one another. In addition, the ulna head section and the radius head section 5; 6 are each connected independently of one another to the distal end 4 of the body section 2 via a corresponding neck section 51; 61.

The body section 2, the ulna head section 5 and the radius head section 5; 6 are each provided with a number of threaded holes 7 for receiving bone securing elements 8, wherein the body section 2 in addition has an elongate compression hole 9.

The fracture fixation plate 1 has a bone contact surface 10 and an opposing surface 11, wherein the bone contact surface 10 is designed such that it is spherically convex in the region of the ulna head section 5 and the bone contact surface 10 is designed such that it is convexly cylindrically curved in the region of the radius head section 6.

By the convex design of the bone contact surface 10 in the region of the ulna head section and the radius head section 5; 6, both bone contact surfaces 10 have a bulging form, wherein the bone contact surface 10 is designed such that it is spherically convex in the region of the ulna head section 5 and cylindrically curved in the region of the radius head section 6. Here, the convexly curved bone contact surface 10, in the region of the radius head section 6, has a curvature which extends from the distal end 4 of the elongated body section 2 to a distal end of the radius head section 6.

The bone contact surface 10, in the region of the ulna head section 5, is substantially trapezoidal, wherein the longer base of the trapezoid comes to lie distally. The bone contact surface 10, in the region of the radius head section 6, is substantially rectangular, wherein one of the two shorter sides of the rectangle comes to lie distally.

Between the ulna head section 5 and the radius head section 6 and the associated neck sections 51; 61, a notch 12 is formed, which is dimensioned/formed in such a manner that the flexor pollicis lungus (FPL) tendon can be accommodated therein.

This notch 12 is designed such that it is V-shaped and continuously widens in the distal direction. Measured in the plane of the plate, the notch 12 has an area F_{E} of 30 mm². The total area F_{TOT} of the fracture fixation plate 1, including the notch 12 and the threaded holes 7, is 630 mm².

In a broader design of the fracture fixation plate, the total area F_{TOT} is 686 mm² and the area F_{E} of the notch is 35 mm².

Furthermore, the threaded holes 7 arranged in the body section 2 each comprise a non-rotatable, positive-locking threaded hole insert 13 which is made of a softer material than the material of the bone fixation plate 1. Analogously, the threaded holes 7 arranged in the ulna head section 5 and in the radius head section 6 also each comprise a non-rotatable, positive-locking threaded hole insert 13 made of a softer material then the material of the bone fixation plate 1.

Although there are various embodiments of the present invention as described above, they should be understood in such a way that the different features can be used both individually and also in any combination.

The invention is therefore not simply limited to the aforementioned, particularly preferred embodiments.

1-15. (canceled)

16: A fracture fixation plate for application to the volar surface of a distal radius, said fracture fixation plate comprising an elongated body section having a free proximal end and a distal end, wherein an ulna head section and a radius head section connect to the distal end and diverge distally from one another, wherein the ulna head section and the radius head section are each independently connected to the distal end of the body section via corresponding neck sections, wherein the body section and the ulna head section and the radius head section are each provided with a number of threaded holes for receiving bone securing elements, wherein the body section is also provided with an elongate compression hole, wherein the fracture fixation plate has a bone contact surface and an opposing surface, and wherein:
a) the bone contact surface is convex in a region of the ulna head section; and
b) the bone contact surface is convex in a region of the radius head section.

17: The fracture fixation plate according to claim 16, wherein the bone contact surface is spherically convex in the region of the ulna head section.

18: The fracture fixation plate according to claim 16, wherein the bone contact surface is cylindrically curved in the region of the radius head section.

19: The fracture fixation plate according to claim 16, wherein the bone contact surface is substantially trapezoidal in shape in the region of the ulna head section, the substantially trapezoidal shape being defined, in part, by a longer parallel base, and wherein the longer parallel base lies distally.

20: The fracture fixation plate according to claim 16, wherein the bone contact surface is substantially rectangular in shape in the region of the radius head section, the substantially rectangular shape being defined, in part, by two shorter sides, and wherein one of the two shorter sides lies distally.

21: The fracture fixation plate according to claim 16, wherein a notch is formed between the ulna head section and the radius head section, and wherein the corresponding neck sections are dimensioned and arranged such that a flexor pollicis lungus tendon is accommodatable therein.

22: The fracture fixation plate according to claim 21, wherein the notch is V-shaped.

23: The fracture fixation plate according to claim 21, wherein the notch, measured in the plane of the fracture fixation plate, has the area F_{E} which is at most 10% of the total area F_{TOT} of the fracture fixation plate, including the notch and holes in the facture fixation plate.

24: The fracture fixation plate according to claim 21, wherein, measured in the plane of the fracture fixation plate, the notch has the area F_{E} which is at least 10 mm².

25: The fracture fixation plate according to claim 21, wherein, measured in the plane of the fracture fixation plate, the notch has the area F_{E} which is at most 60 mm².

26: The fracture fixation plate according to claim 16, wherein the body section has at least one threaded hole with a non-rotatable, positive-locking threaded hole insert made of a softer material than the material of the fracture fixation plate.

27: The fracture fixation plate according to claim 16, wherein the ulna head section and the radius head section each have a threaded hole with a non-rotatable, positive-locking threaded hole insert made of a softer material than the material of the fracture fixation plate.

28: The fracture fixation plate according to claim 16, wherein the ulna head section has three threaded holes, each including a non-rotatable, positive-locking threaded hole insert made of a softer material than the material of the fracture fixation plate.

29: The fracture fixation plate according to claim 16, wherein the radius head section has two or three threaded holes, each including a non-rotatable, positive-locking threaded hole insert made of a softer material than the material of the fracture fixation plate.

30: The fracture fixation plate according to claim 16, wherein the convex bone contact surface has a curvature in the region of the radius head section, and wherein the curvature extends from the distal end of the elongated body section to a distal end of the radius head section.
